(19) United States
(12) Patent Application Publication
(10) Pub. No.: US 2016/0022710 A1
(43) Pub. Date: Jan. 28, 2016

(54) PRENATAL AND LACTATION SUPPLEMENTS TO ENHANCE CENTRAL NERVOUS SYSTEM DEVELOPMENT OF OFFSPRING

(71) Applicant: ABBOTT LABORATORIES, Abbott Park, IL (US)

(72) Inventors: Chron-Si Lai, Blacklick, OH (US); Tama Bloch, Columbus, OH (US); Christina Sherry, Westerville, OH (US); Maria Ramirez Gonzalez, Granada (ES); Matthew Kuchan, Westerville, OH (US); Gary Katz, Columbus, OH (US); Elena Oliveros Delgado, Granada (ES); Angela Santos, Granada (ES)

(73) Assignee: ABBOTT LABORATORIES, Abbott Park, IL (US)

(21) Appl. No.: 14/774,745

(22) PCT Filed: Mar. 13, 2014
(86) PCT No.: PCT/US2014/026239

§ 371 Date: Sep. 11, 2015

Related U.S. Application Data
(60) Provisional application No. 61/779,265, filed on Mar. 13, 2013.

(51) Int. Cl. A61K 31/685; A61K 31/202; A61K 31/047; A23L 1/29; A61K 31/015; A61K 31/045; A61K 9/48; A61K 31/355; A61K 31/593

(52) U.S. Cl. CPC A61K 31/685; A61K 31/355; A61K 31/202; A61K 31/047; A61K 31/593; A61K 31/015; A61K 31/045; A61K 9/48; A23L 1/296; A23V 2002/00

(57) ABSTRACT

Disclosed are prenatal and lactation supplements for pregnant women and lactating women, which include a combination of RRR-alpha-tocopherol, docosahexaenoic acid (DHA), trans-lutein, phospholipids, and at least one nuclear receptor activating ligand other than RRR-alpha-tocopherol, DHA, and trans-lutein. The supplements may enhance central nervous system development in a fetus or breast-feeding newborn infant.

### CROSS REFERENCE TO RELATED APPLICATIONS

This application claims priority to and any benefit of U.S. Provisional Application No. 61/779,265, filed Mar. 13, 2013, the entire content of which is incorporated herein by reference.

### FIELD

The present disclosure relates to prenatal and lactation supplements for pregnant women and lactating women. The supplements comprise a combination of RRR-alpha-tocopherol, docosahexaenoic acid (DHA), trans-lutein, phospholipids, and at least one nuclear receptor activating ligand other than RRR-alpha-tocopherol, DHA, or trans-lutein. The disclosure further relates to methods of administering the prenatal and lactation supplements to pregnant women and lactating women to enhance central nervous system development in the fetus and nursing newborns.

### BACKGROUND

Maturation of the central nervous system (CNS), including the brain and eyes, is a key developmental area for the fetus and newborn. Often times, pregnant women take prenatal supplements to provide additional nutrients to the developing fetus in utero. Similarly, lactating women who breast feed their newborn may take lactation supplements to provide additional nutrients to the newborn through breast milk.

### SUMMARY

The present disclosure relates to prenatal and lactation supplements for pregnant women and lactating women. The present disclosure also relates to methods for enhancing CNS development in a fetus or breastfed newborn. It has surprisingly been found that specific combinations of the CNS maturation enhancers as described herein may be passed from a mother to her child in utero or via breast milk, such that the CNS maturation of her fetus or breastfed newborn may be improved. The CNS maturation enhancers may include, but are not limited to, DHA, RRR-alpha-tocopherol, and trans-lutein.

Without wishing to be bound by theory, Applicants believe that a combination of DHA, RRR-alpha-tocopherol, and trans-lutein stimulates fetal and newborn infant CNS maturation by upregulating the expression of certain genes. Certain nuclear receptors bind these compounds and the resultant activated nuclear receptor may translocate into the cell nucleus. Applicants believe that the activated nuclear receptor may bind to an activated co-factor to form a dimer. The resulting dimer may alter gene expression and thus increase CNS maturation. If a dimer is not formed, it is believed that the activated nuclear receptor may not be highly functional in relation to altering gene expression. For these reasons at a minimum, the present supplements further comprise at least one nuclear receptor activating ligand other than RRR-alpha-tocopherol, DHA, or trans-lutein.

RRR-alpha-tocopherol, DHA, and trans-lutein are lipid soluble CNS maturation enhancers and it is reported that they are present in the circulation in the form of lipoproteins. In order for the developing fetus to acquire these CNS maturation enhancers, they may need to be taken up by the placenta. Indeed, it is suggested in the literature that a lack of placental uptake of high density lipoproteins may lead to defective neural tube development. In order for the nursing infant to acquire these CNS maturation enhancers, the enhancers may first be absorbed by the mammary gland. It is believed that the mammary gland uptakes these enhancers in the form of high density lipoproteins (HDL) and then excretes the enhancers into the breast milk. For these reasons, the present supplements further include phospholipids, such as those derived from lecithin.

Since it is reported that most of the alpha-tocopherol in circulation is in the lipoproteins, Applicants believe that non-RRR-alpha-tocopherol may compete with RRR-alpha-tocopherols for presence within the circulating lipoproteins. Consequently, Applicants believe that non-RRR-alpha-tocopherol competes with RRR-alpha-tocopherol for absorption into the mammary gland or into the placenta. Thus, the present supplements may be substantially free of, or free of, non-RRR-alpha-tocopherol.

Accordingly, in one embodiment, a supplement of the present disclosure comprises, in terms of the amount of ingredient per kilogram of body weight of a pregnant or lactating woman per day of: from about 0.2 mg to about 100 mg of RRR-alpha-tocopherol; no more than about 10 mg of non-RRR-alpha-tocopherol; from about 3 mg to about 50 mg of DHA; from about 40 μg to about 500 μg of trans-lutein; and from about 0.9 mg to about 100 mg of lecithin. In addition, the supplement comprises at least one nuclear receptor activating ligand other than RRR-alpha-tocopherol, DHA, or trans-lutein.

In some embodiments, the prenatal and lactation supplement is in a unit dose form, such as a capsule or softgel.

In some embodiments, the present disclosure is directed to a method for enhancing CNS development in a fetus or a breastfed newborn, the method comprising the step of orally administering to a pregnant or lactating woman a daily dose of a supplement comprising, in terms of the amount of ingredient per kilogram of body weight per day of: from about 0.2 mg to about 100 mg of RRR-alpha-tocopherol; no more than about 10 mg of non-RRR-alpha-tocopherol; from about 3 mg to about 50 mg of DHA; from about 40 μg to about 500 μg of trans-lutein; from about 0.9 mg to about 100 mg of lecithin; and at least one of nuclear receptor activating ligand other than RRR-alpha-tocopherol, DHA, or trans-lutein.

Accordingly, the prenatal and lactation supplements and methods of the present disclosure may offer a therapeutic or intervention option for a pregnant woman or a lactating woman that may contribute to the enhanced development of the CNS in the fetus or breastfed newborn.

### DETAILED DESCRIPTION

“Prenatal and lactation supplement” as used herein refers to a unit dose of the supplements presently described. The supplement may be in any suitable form including, but not limited to, capsules, softgels, and the like. The prenatal and lactation supplement may further comprise vitamins, minerals, and other ingredients that are beneficial to a pregnant or lactating woman.

“Softgel” and “capsule” are used interchangeably herein to mean a material that is enclosed by an edible film or gel.

“Cognitive performance” as used herein, unless otherwise specified, refers to the learning, thinking, and memory functions (i.e., memory acquisition, memory retention, and memory recall) of the brain. Accordingly, the term “improving cognitive performance” as used herein, unless otherwise specified, refers to improving the learning, thinking, and/or memory (memory acquisition, memory retention, and memory recall) functions of an infant.

“Newborn” is used interchangeably herein with “newborn infant” and “infant.” As used herein a “newborn” means, unless otherwise specified, infants less than about 3 months of age, including infants from zero to about 2 weeks of age. As used herein a “term infant” refers to individuals born at or beyond 37 weeks of gestation, unless otherwise specified.

All percentages, parts, and ratios as used herein are by weight of the total product, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the prenatal and lactation supplement of the present disclosure may also be substantially free of any ingredient or feature described herein, provided that the remaining formula still contains all of the required ingredients or features as described herein. In this context, and unless otherwise specified, the term “substantially free” means that the composition contains less than a functional amount of the optional ingredient, typically less than 1%, including less than 0.5%, including less than 0.1%, and also including zero percent, by weight of such optional ingredient.

The prenatal and lactation supplements may comprise, consist of, or consist essentially of the elements of the products as described herein, as well as any additional or optional element described herein or otherwise useful in prenatal and/or lactation product applications.

The supplements and methods disclosed herein are directed to prenatal and lactation supplements for pregnant women or lactating women. The supplements include a combination of DHA, RRR-alpha-tocopherol, no more than 10 mg of non-RRR-alpha-tocopherol chiral isomers, trans-lutein, phospholipids from lecithin, and at least one nuclear receptor activating ligand other than trans-lutein, RRR-alpha-tocopherol, or DHA. These and other elements or features of the various embodiments are described in detail hereafter.

### A. Docosahexaenoic Acid (DHA)

The prenatal and lactation supplements of the present disclosure comprise docosahexaenoic acid (DHA), which is a long chain polyunsaturated fatty acid (LCPUFA).

DHA is added among other reasons, because a newborn infant may be unable to synthesize sufficient levels of DHA to meet its growth needs. Therefore, the newborn infant may rely on DHA from breast milk. It is reported that maternal diet DHA content can affect breast milk DHA level. As such, the supplements of the present disclosure are believed to increase breast milk DHA level to promote newborn infant brain and eye development.

Without wishing to be bound by theory, it is believed that DHA acts as follows. DHA activates retinoid X receptors (RXR), which may upregulate gene expression. This may explain why dietary DHA enhances brain phospholipid synthesis. Since phospholipids are a major component of myelin and neuronal cell membranes, it is believed that the higher brain phospholipid content is reflective of advanced maturation of the CNS in the developing fetus or growing child. Thus, it is for these reasons at a minimum that DHA is believed to play an important role in the CNS development of the fetus or newborn.

DHA is included in the prenatal and lactation supplements as a free fatty acid, in ethyl ester form, in triglyceride form, or in combinations thereof. In some embodiments, DHA in triglyceride form is preferred.

The supplements of the present disclosure may provide a daily dose of DHA in terms of the amount of DHA dosed per kilogram of body weight (bw) of the pregnant woman or lactating woman per day of from about 3 mg to about 50 mg of DHA. In certain embodiments, the supplements provide a daily dose of DHA of from about 5 mg DHA/kg bw/day to about 45 mg DHA/kg bw/day, from about 10 mg DHA/kg bw/day to about 40 mg DHA/kg bw/day, or from about 20 mg DHA/kg bw/day to about 30 mg DHA/kg bw/day. A suitable form of DHA for use in the supplements disclosed herein is available from Martek Biosciences Corporation of Columbia, Md.

### B. RRR-Alpha-Tocopherol

As used herein, the term “RRR-alpha-tocopherol” refers to both exogenous sources and inherent sources of RRR-alpha-tocopherol and RRR-alpha-tocopherol acetate that may be present in the present prenatal and lactation supplement. Inherent sources are supplement components, such as oils or fat, which inherently comprise RRR-alpha-tocopherol. Exogenous sources of RRR-alpha-tocopherol include RRR-alpha-tocopherol that is added to the prenatal and lactation supplement not as part of another component.

Tocopherols, which are generically referred to as vitamin E, have the following general structure:

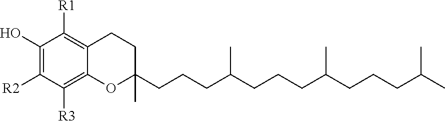

Tocopherols are available in four forms: alpha, beta, gamma, and delta, which differ in the number and position of the methyl groups on the chroman ring as shown in Table 1.

TABLE 1

| Compound | R1 | R2 | R3 |
|---|---|---|---|
| alpha-tocopherol | Me | Me | Me |
| beta-tocopherol | Me | H | Me |
| gamma-tocopherol | H | Me | Me |
| delta-tocopherol | H | H | Me |

Tocopherols can also exist in a number of stereoisomeric forms depending on the chirality of the phytyl tail. Of the alpha-tocopherols, RRR-alpha-tocopherol (also referred to as “natural vitamin E”) has the greatest biological activity and is reported to be the dominant form of the alpha-tocopherol in the brain. RRR-alpha-tocopherol is a single stereoisomer whereas synthetic vitamin E (all-rac-alpha-tocopherol or tocopherol acetate) is an equimolar mixture of eight isomers, only one of which is RRR-alpha-tocopherol.

Applicants have surprisingly found that regardless of the form of alpha-tocopherol in an infant's diet, the dominant form of alpha-tocopherol in the infant's brain is RRR-alpha-tocopherol. This is surprising indeed given that typical infant formulas are fortified with synthetic alpha-tocopherol. This finding strongly suggests that the other seven chiral isomers may be absorbed at a lower rate by the brain and/or oxidized by the brain at a faster rate.

It was surprisingly discovered that there is a correlation between the levels of RRR-alpha-tocopherol and the levels of cholesterol and glutamate in the brain. Cholesterol and glutamate may be important for brain and CNS development for the following reasons at a minimum. For example, cholesterol is a major component of neuronal cell membranes and myelin, whereas glutamate is a neurotransmitter which is found to stimulate neuron outgrowth and branching. Without wishing to be bound by theory, it is believed that this correlation may result from the following. Alpha-tocopherol binds tocopherol associated protein (TAP) to form a TAP-alpha-tocopherol complex. The complex is shown to translocate into the cell nucleus. Applicants believe that of the alpha-tocopherols, TAP preferably binds RRR-alpha-tocopherol and that the resulting complex protects the RRR-alpha-tocopherol from being metabolized or oxidized. Thus, based upon the forgoing, a higher level of non-RRR-alpha-tocopherol may compete with RRR-alpha-tocopherol for TAP and as such may compromise the beneficial effects of RRR-alpha-tocopherol. As discussed previously, alpha-tocopherol, which may be present in the maternal HDL, is taken up by the placenta and mammary gland. Thus, to maximize the beneficial effects of RRR-alpha-tocopherol, the level of non-RRR-alpha-tocopherol in the prenatal and/or lactation supplements is limited.

The supplements of the present disclosure may provide a daily dose of RRR-alpha-tocopherol in terms of the amount of RRR-alpha-tocopherol dosed per kilogram of body weight of the pregnant woman or lactating woman per day of from about 0.2 mg to about 100 mg of RRR-alpha-tocopherol. In certain embodiments, the supplements provide a daily dose of RRR-alpha-tocopherol of from about 0.5 mg RRR-alpha-tocopherol/kg bw/day to about 90 mg RRR-alpha-tocopherol/kg bw/day, from about 5 mg RRR-alpha-tocopherol/kg bw/day to about 80 mg RRR-alpha-tocopherol/kg bw/day, from about 10 mg RRR-alpha-tocopherol/kg bw/day to about 75 mg RRR-alpha-tocopherol/kg bw/day, from about 25 mg RRR-alpha-tocopherol/kg bw/day to about 50 mg RRR-alpha-tocopherol/kg bw/day, or from about 30 mg RRR-alpha-tocopherol/kg bw/day to 40 mg RRR-alpha-tocopherol/kg bw/day.

The supplements of the present disclosure may provide less than about 10 mg of non-RRR-alpha-tocopherol per kilogram of body weight of a pregnant woman or a lactating woman per day. In certain embodiments, the supplements provide no more than about 5 mg non-RRR-alpha-tocopherol/kg bw/day, no more than about 2.5 mg non-RRR-alpha-tocopherol/kg bw/day, no more than about 1 mg non-RRR-alpha-tocopherol/kg bw/day, no more than about 0.5 mg non-RRR-alpha-tocopherol/kg bw/day, no more than about 0.1 mg non-RRR-alpha-tocopherol/kg bw/day, or no more than about 0.01 mg non-RRR-alpha-tocopherol/kg bw/day. In certain embodiments, the supplement is substantially free of non-RRR-alpha-tocopherol. In certain embodiments, the supplement is free of non-RRR-alpha-tocopherol.

A suitable form of RRR-alpha-tocopherol for use in the supplements disclosed herein is Novatol 5-67S, which is available from Archer Daniels Midland of Decatur, Ill.

In certain embodiments, the DHA and RRR-alpha-tocopherol are present in the supplement at a weight ratio of from about 5:1 to about 15:1 (DHA:RRR-alpha-tocopherol). In certain embodiments, the DHA and RRR-alpha-tocopherol are present in the supplement at a weight ratio of from about 7:1 to about 12:1, or from about 8:1 to about 10:1.

### C. Trans-Lutein

The prenatal and lactation supplements of the present disclosure comprise trans-lutein, which is surprisingly found to be the predominant carotenoid in the majority of neonatal brain samples studied by the Applicants. This is surprising because although the level of the carotenoid lycopene in human breast milk is equal to or higher than that of lutein, very few neonatal brain samples are found to contain detectable levels of lycopene.

It is believed that during fetal CNS development, neural stem cells differentiate into progenic cells, which then differentiate into neuron and glial cells. It is reported that trans-lutein and beta-cryptoxanthin activate the retinoic acid activated receptor (RAR), that peroxisome proliferator-activated receptor (PPAR) gamma forms heterodimers with RAR, and PPAR gamma is reported to promote neural progenic cell differentiation. Thus, by activating RAR, trans-lutein is believed to influence CNS development. It is also know that RAR forms heterodimers with retinoid X receptor (RXR). It is reported that DHA activate RXR to stimulate neural stem cell differentiation. Thus, it is most likely that the potency of DHA on CNS development will be drastically enhanced by lutein.

It has further been discovered that the trans-lutein level in the infant brain correlates with levels of gamma amino butyric acid (GABA). GABA is believed to stimulate new born animal CNS development. Thus, for these reasons at a minimum, it is believed that trans-lutein stimulates fetal and postnatal CNS development.

It is reported that maternal plasma lutein can cross the placenta into the fetus. Thus, based upon the foregoing, Applicants believe that fetal and breastfed newborn CNS development can be enhanced by providing a nutritional supplement comprising trans-lutein to a pregnant woman and a lactating woman.

The supplements of the present disclosure may provide a daily dose of trans-lutein in terms of the amount of trans-lutein dosed per kilogram of body weight of the pregnant or lactating woman per day of from about 40 μg to about 500 μg. In certain embodiments, the supplements provide a dose of trans-lutein of from about 50 μg trans-lutein/kg bw/day to about 400 μg trans-lutein/kg bw/day, from about 100 μg trans-lutein/kg bw/day to about 300 μg trans-lutein/kg bw/day, or from about 150 μg trans-lutein/kg bw/day to about 250 μg trans-lutein/kg bw/day.

As used herein, “trans-lutein” refers to a compound having the following structure:

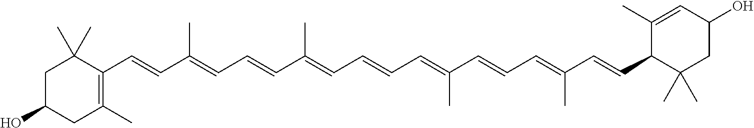 [SMILES: CC1=CC(O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

Trans-lutein may be obtained from any suitable material source for use in the present prenatal and lactation supplements. An exemplary source of trans-lutein that may be used in the supplements described herein is FloraGlo® from Kemin Industries, Inc. (Des Moines, Iowa).

### D. Phospholipid

A phospholipid is included in the supplements of the present disclosure for at least the following reasons. First, inclusion of phospholipid in the present prenatal and lactation supplements may enhance maternal trans-lutein absorption. Without wishing to be bound by theory, it is believed that trans-lutein is incorporated into mixed micelles before it can be absorbed into the bloodstream. The phospholipid is digested to form lysophospholipids and fatty acids. Lysolecithin has a much higher solubility than free fatty acids, and thus, lysolecithin can facilitate the formation of mixed micelles. In addition, lysolecithin can more effectively “ferry” lutein and RRR-alpha-tocopherol into the mixed micelles than free fatty acids from triglyceride digestion.

Second, the inclusion of phospholipids may enhance the bioavailability of lutein and RRR-alpha-tocopherol. It is believed that most of the circulating RRR-alpha-tocopherol and lutein is present in the lipoprotein fraction. It is reported that HDL comprising RRR-alpha-tocopherol and lutein is taken up by the placenta and mammary gland. Thus, it follows that in order to enhance the delivery of RRR-alpha-tocopherol and lutein to the fetus via the placenta or to the infant via breast milk, it is desirable to increase the maternal HDL level and to enhance the loading of lutein and RRR-alpha-tocopherol onto HDL particles. Dietary lecithin has been reported to positively affect HDL levels, presumably by providing building blocks in the liver for the HDL. Thus, inclusion of phospholipid in the prenatal and lactation supplements is believed to enhance the bioavailability of lutein and RRR-alpha-tocopherol.

Third, the inclusion of phospholipids may enhance the bioavailability of DHA. It is reported that most of the DHA in the breast milk is in phospholipid form. Thus, the level of DHA in the maternal HDL can affect the level of placental DHA uptake as well as the DHA level in breast milk. Dietary phospholipids may increase the level of lutein and tocopherol delivered to the maternal liver and thereby increase the level of DHA synthesis. Thus, it is believed that inclusion of lecithin in the present supplement will enhance the bio-availability of DHA to the fetus or breast-feeding infant.

The supplements of the present disclosure may provide a daily dose of lecithin in terms of the amount of lecithin dosed per kilogram of body weight of the pregnant or lactating woman per day of from 0.9 mg to about 100 mg of lecithin. In certain embodiments, the supplements provide a dose of lecithin of from about 1 mg lecithin/kg bw/day to about 90 mg lecithin/kg bw/day, from about 10 mg lecithin/kg bw/day to about 80 mg lecithin/kg bw/day, from about 25 mg lecithin/kg bw/day to about 75 mg lecithin/kg bw/day, or from about 35 mg lecithin/kg bw/day to about 50 mg lecithin/kg bw/day. Lecithin derived from vegetable oils or egg yolk both contain a high level of phospholipids and, thus, are suitable for this application. However, due to pricing and concern of allergens, vegetable oil lecithin may, in some instances, be a preferred source of phospholipids.

In certain embodiments, the DHA and the lecithin are present in the supplement at a weight ratio of from about 1:1 to about 5:1 (DHA:lecithin). In certain embodiments, the DHA and lecithin are present in the supplement at a weight ratio of from about 1.5:1 to about 4:1, or from about 2:1 to about 3:1.

### E. Nuclear Receptor Activating Ligand

The prenatal and lactation supplements of the present disclosure comprise at least one nuclear receptor activating ligand other than RRR-alpha-tocopherol, DHA, or trans-lutein. Without wishing to be bound by theory, it is believed that the nuclear receptor activating ligands may enhance CNS development as follows.

Trans-lutein is suggested to stimulate fetal CNS maturation by activating Retinoic activated receptor (RAR), which in turn is believed to promote neural progenic cell differentiation and thus CNS development. It is believed that RAR form heterodimers with vitamin D receptors (VDR) to exert its full effect on gene expression. VDR may itself be activated by 1, 25 OH vitamin D. Thus, vitamin D and its metabolites are nuclear receptor activating ligands that may enhance CNS development and as such may be included in the present supplements.

DHA is suggested to activate RXR which stimulates neural stem cell differentiation, and thus, fetal CNS development. RXR may form a dimer with activated VDR. 1, 25 OH Vitamin D is a ligand that activates the VDR. Thus, vitamin D and its metabolites may theoretically enhance the effect of DHA on CNS maturation. RXR is suggested to require an activated co-factor to exert its full effect on gene expression. RXR is suggested to form a dimer with RAR. Beta-cryptoxanthin is suggested to activate RAR. Theoretically, beta-cryptoxanthin will significantly enhance the potency of DHA as well. It is also known that VDR and RXR form heterodimers. Theoretically, there should be synergy between Vitamin D and DHA on CNS maturation as well.

It is reported that alpha-tocopherol binds tocopherol association protein (“TAP”) and the resulting complex translocates into the cell nucleus. This finding suggests that TAP is a nuclear receptor that regulates gene expression. Applicants' discovery that infant brain alpha-tocopherol correlates with cholesterol and glutamate levels suggests that alpha-tocopherol upregulates the genes responsible for cholesterol and glutamate synthesis in order to stimulate CNS development. Most of the nuclear receptors require an activated co-factor. Thus, it is very likely that TAP may need a ligand that activates VDR, PPAR, RAR, RXR, or pregnane X receptor (PXR) to exert its full effect.

The metabolites of Vitamin A and beta-carotene are reported to activate both RXR and RAR, and thus, theoretically, vitamin A or beta-carotene can enhance the beneficial effect of lutein and DHA, and perhaps RRR-alpha-tocopherol as well.

Non-limiting examples of useful VDR, PPAR, RXR, RAR, or PXR nuclear receptor activating ligands other than RRR-alpha-tocopherol, DHA, or trans-lutein may include, but are not limited to: vitamin D (e.g., vitamin D₂, vitamin D₃), vitamin D metabolites (e.g., calciferol, cacidiol, calcitriol); beta-carotene; beta-cryptoxanthin; zeaxanthin; vitamin A; vitamin A metabolites; phospholipids; nucleotides; and combinations thereof. In certain embodiments, the vitamin D metabolites are selected from the group of: calciferol; calcidiol; calcitriol; and combinations thereof.

The nuclear receptor activating ligands may be present in the supplement at a useful level as determined by one skilled in the art. Moreover, one skilled in the art may select additional ingredients to include in the prenatal and lactation supplements of the present disclosure.

### F. Methods of Use

The methods of the present disclosure include a method to stimulate fetus or new born infant CNS development comprising the step of orally administering the present prenatal and lactation supplements to a pregnant or lactating woman. The prenatal and lactation supplements of the present disclosure may provide a daily dose in terms of the amount of ingredient per kilogram of body weight of a pregnant or lactating woman per day of: from about 0.3 mg to about 100 mg of RRR-alpha-tocopherol; no more than about 10 mg of non-RRR-alpha-tocopherol; from about 3 mg to about 50 mg of DHA; from about 40 μg to about 500 μg of trans-lutein; from about 0.9 mg to about 100 mg of lecithin; and at least one nuclear receptor activating ligand other than RRR-alpha-tocopherol, DHA, or trans-lutein. It should be understood that any of the previously described embodiments of the prenatal and lactation supplement may be utilized in the methods of the present disclosure.

The prenatal and lactation supplement may provide critical nutrients needed for CNS development in the fetus and breast-feeding newborns.

In addition to enhancing CNS development, the prenatal and lactation supplements can be administered to improve cognitive performance in a breast-feeding newborn infant. Particularly, the combination of RRR-alpha-tocopherol and DHA may improve general cognition by enhancing memory acquisition, memory retention, and memory recall that contributes to the cognitive functions of learning, thinking, and memory.

The prenatal and lactation supplements as described herein can be administered to pregnant or lactating women that are “in need thereof” that is, to specific women whose offspring would specifically benefit by administration of the prenatal and lactation supplement. For example, a specific woman may be “in need of” the prenatal and lactation supplements as described herein if their offspring are susceptible to (i.e., genetically predisposed, have a family history of, and/or having symptoms of the disease or condition) neurodegenerative diseases or other diseases and conditions that can impair or otherwise reduce cognition generally or specific aspects of cognition.

### Examples

Prenatal and lactation supplements according to the present disclosure may be prepared in accordance with manufacturing methods well known in the nutrition industry.

Table 2 provides examples of supplements according to the present disclosure. Each of the ingredient amounts should be considered to be preceded by the term “about.”

TABLE 2

| Ingredient | Example 1 | Example 2 | Example 3 |
|---|---|---|---|
| ¹Fish oil (DHA 4T1400) | 472 | 472 | 472 |
| ²Novatol 5-67S | 31.5 | 31.5 | 31.5 |
| ³Floraglo (20% lutein crystal) | 33 | 33 | 33 |
| ⁴Soy lecithin | 60 | 60 | 60 |
| ⁵Beta-carotene (20% beta crystal) | 16 | 16 | 16 |
| Beta-cryptoxanthin | — | 5 | — |
| Calcidiol | — | — | 0.005 |

¹from Ocean Nutrition (Nova Scotia, Canada)
²from Archer Daniels Midland (Decatur, Illinois, USA)
³from Kemin Industries, Inc. (Des Moines, Iowa, USA)
⁴from Archer Daniels Midland (Decatur, Illinois, USA)
⁵from DSM (Delft, The Netherlands)

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions and processes, and illustrative examples shown and described. Accordingly, departures may be made from such details without departing from the spirit or scope of the general inventive concept.

1. A supplement for pregnant and lactating women, comprising in terms of the amount of ingredient per kilogram of body weight of the woman per day of: from about 0.2 mg to about 100 mg of RRR-alpha-tocopherol; no more than about 10 mg of non-RRR-alpha-tocopherol; from about 3 mg to about 50 mg of docosahexaenoic acid; from about 40 μg to about 500 μg of trans-lutein; from about 0.9 mg to about 100 mg of lecithin; and at least one nuclear receptor activating ligand other than RRR-alpha-tocopherol, docosahexaenoic acid and trans-lutein.

2. The supplement according to claim 1, wherein the nuclear receptor activating ligand is selected from the group of: vitamin D; vitamin D metabolites; beta carotene; beta-cryptoxanthin; zeaxanthin; vitamin A; vitamin A metabolites; phospholipids; nucleotides; and combinations thereof.

3. The supplement according to claim 2, wherein the vitamin D metabolites are selected from the group of: calciferol; cacidiol; calcitriol; and combinations thereof.

4. The supplement according to claim 1, wherein the composition is in the form of a capsule or softgel.

5. The supplement according to claim 1, wherein the docosahexaenoic acid and the RRR-alpha-tocopherol are present in the supplement at a weight ratio of from about 5:1 to about 15:1.

6. The supplement according to claim 1, wherein the docosahexaenoic acid and the lecithin are present in the supplement at a weight ratio of from about 1:1 to about 5:1.

7. The supplement according to claim 1, wherein the docosahexaenoic acid is in the form of a triglyceride.

8. The supplement according to claim 1, wherein the supplement comprises no more than about 5 mg of non-RRR-alpha-tocopherol.

9. The supplement according to claim 8, wherein the supplement comprises no more than about 0.1 mg of non-RRR-alpha-tocopherol.

10. The supplement according to claim 8, wherein the supplement is substantially free of non-RRR-alpha-tocopherol.

11. A method for enhancing central nervous system development in a fetus or breastfed newborn, the method comprising the step of orally administering to a pregnant or lactating woman a daily dose of a supplement comprising in terms of the amount of ingredient per kilogram of body weight of the woman per day of: from about 0.3 mg to about 100 mg of RRR-alpha-tocopherol; no more than about 10 mg of non-RRR-alpha-tocopherol; from about 3 mg to about 50 mg of DHA; from about 40 μg to about 500 μg of trans-lutein; from about 0.9 mg to about 100 mg of lecithin; and at least one nuclear receptor activating ligand other than RRR-alpha-tocopherol, DHA, and trans-lutein.

12. The method according to claim 11, wherein the nuclear receptor activating ligand is selected from the group of: vitamin D; vitamin D metabolites; beta carotene; beta-cryptoxanthin; zeaxanthin; vitamin A; vitamin A metabolites; phospholipids; nucleotides; and combinations thereof.

13. The method according to claim 12, wherein the vitamin D metabolites are selected from the group of: calciferol; cacidiol; calcitriol; and combinations thereof.

14. The method according to claim 11, wherein the composition is in the form of a capsule or softgel.

15. The supplement according to claim 11, wherein the docosahexaenoic acid and the RRR-alpha-tocopherol are present in the supplement at a weight ratio of from about 5:1 to about 15:1.

16. The method according to claim 11, wherein the docosahexaenoic acid and the lecithin are present in the supplement at a weight ratio of from about 1:1 to about 5:1.

17. The method according to claim 11, wherein the docosahexaenoic acid is in the form of a triglyceride.

18. The method according to claim 11, wherein the supplement comprises no more than about 5 mg of non-RRR-alpha-tocopherol.

19. The method according to claim 18, wherein the supplement comprises no more than about 0.1 mg of non-RRR-alpha-tocopherol.

20. The method according to claim 19, wherein the supplement is substantially free of non-RRR-alpha-tocopherol.
